(19) United States
(12) Patent Application Publication
(10) Pub. No.: US 2013/0281833 A1
(43) Pub. Date: Oct. 24, 2013

(54) DEVICE AND METHOD FOR DIAGNOSING LUNG CANCER USING ELECTROMAGNETIC WAVE

(71) Applicant: KOREA INSTITUTE OF SCIENCE AND TECHNOLOGY, Seoul (KR)

(72) Inventors: Se Yun KIM, Seoul (KR); Ji Hyun JUNG, Seoul (KR)

(21) Appl. No.: 13/866,142

(22) Filed: Apr. 19, 2013

(30) Foreign Application Priority Data
Apr. 19, 2012 (KR) 10-2012-0041088

(51) Int. Cl. A61B 5/00

(52) U.S. Cl. CPC A61B 5/4887
USPC 600/424

(57) ABSTRACT

The present invention relates to a device and method for diagnosing lung cancer using electromagnetic wave. The device includes a receiving part to receive magnetic wave generated from an electromagnetic wave transmitter ingested through human oral cavity and moving along with digestive organs; a measuring part to measure amplitude and phase information of the received electromagnetic wave; and a controller to diagnose the pulmonary tumor based on the above measurement results and the location information from the transmitter.

### CROSS-REFERENCE TO RELATED APPLICATION

Pursuant to 35 U.S.C. §119(a), this application claims the benefit of earlier filing date and right of priority to Korean Application No. 10-2012-0041088, filed on Apr. 19, 2012, the contents of which is incorporated by reference herein in its entirety.

### BACKGROUND OF THE INVENTION

1. Field of the Invention

The present invention relates to a method for determining a location of a pulmonary tumor using electromagnetic wave and a diagnostic device using the method.

2. Background of the Invention

Pulmonary tumor is mutant cells, which includes malignant and benign tumor. A representative one of malignant tumors is lung cancer and even in case of benign tumor, there is a possibility to develop to malignant one, so it is very important to find it early.

Noninvasive medical diagnosis techniques to diagnose a tumor and determine its location includes X-ray, CT (Computed Tomography), and bronchial endoscope diagnosis.

In the X-ray diagnosis technique, arrange a light source and a light sensing plate make a patient located between them, and obtain images of human internal organs by using difference of transmissibility of X-ray. When using this method, 3-dimensional images are displayed on the film 2-dimensionally.

Also in the diagnosis method using CT, transmissibility difference of X-ray is used as like in the above. In the diagnosis technique, arrange X-ray light source and a sensing device and locate a patient between them. And cross sectional images are rearranged by performing image treatment after detecting X-ray intensity and attenuation constant.

Bronchial endoscope diagnosis technique is a method to observe bronchus linked to the lung using the endoscope. The method includes a method using flexuous bronchial endoscope and a method using a stiff bronchial.

### SUMMARY OF THE INVENTION

The techniques using X-ray and CT have a problem that human body is exposed to radiation. In case of the technique using the bronchial endoscope, anesthesia is essential, so that there are some problems that post its procedure time is long and a patient with hypercapnia or sever dyspnea is difficult to receive the procedure. In addition, conventional techniques have also a problem that comes to be vulnerable to external noises in process of reconstructing images through signal treatment of scan data.

Therefore, an objective of the present invention is to make diagnosing tumor and determining its location possible with relatively simple method by solving the above mentioned problems.

In order to achieve the above object, the present invention provides a diagnostic device to determine a position of pulmonary tumor using electromagnetic wave and a method for determining the location using the same.

The device to determine a position of pulmonary tumor using electromagnetic wave may include a receiving part to receive magnetic wave generated from an electromagnetic wave transmitter ingested through human oral cavity and moving along with digestive organs (for example, esophagus), an electromagnetic wave measuring part to measure amplitude and phase information of the received electromagnetic wave, and a controller to determine a position of pulmonary tumor based on the location information from the transmitter.

In addition, the device to determine a position of pulmonary tumor using electromagnetic wave may include also a communication part able to perform mutual communication for sending electromagnetic wave transmission operation control signal to the transmitter or receiving location and state information from the transmitter. For this, the above electromagnetic wave transmitter may include also a communication part for the exchange of information.

The above electromagnetic wave transmitter of the present invention can generate electromagnetic wave according to several frequencies and certain period.

The above receiving part of the device for determining a position of pulmonary tumor using electromagnetic wave according to the present invention may include 5 receiving modules which are contact or located near to the surface of human body and arranged in an array.

The above controller of the device to determine a position of pulmonary tumor according to an example of the present invention can detect volume location linking the above dip to the transmission location with a virtual line and judge it as the location of tumor when a dip is determined from the amplitude information measured from multiple electromagnetic transmission locations.

The above controller according to another example of the present invention can detect volume location linking the above dip to the transmission location with a virtual line and judge it as the location of tumor based on double dip obtained from amplitude information measured in the location of positive off set (+offset) where vertical location of the transmitter is located lower than the receiving module, zero offset (0 offset) where the vertical location is located same to the receiving module, and negative offset (−offset) where the vertical location is located higher that the receiving module and location information of the transmitter.

The method for determining a position of pulmonary tumor using electromagnetic wave according to the present invention may include several steps comprising: a step to receive electromagnetic wave generated from a transmitter ingested through the human oral cavity and moving along with the digestive tract; a step to measure amplitude and phase information from the received electromagnetic wave; and a step to determine the location of tumor based on the above measurement results and location information of the transmitter. In addition, The above method of the present invention may include also a step to send the electromagnetic wave transmission operation control signal, or a step to receive location and status information of the transmitter from it in human body.

The above step to determine a position of pulmonary tumor in the method of the present invention, can detect volume location linking the above dip to the transmission location with a virtual line and judge it as the location of tumor when a dip is determined from the amplitude information measured from multiple electromagnetic transmission locations.

The above step to receive the electromagnetic wave in the method to determine a position of pulmonary tumor according to another example of the present invention can receive electromagnetic wave in several locations including: a positive off set (+offset) where vertical location of the transmitter is located lower than the receiving module; zero offset (0 offset) where the vertical location is located same to the receiving module; and negative offset (−offset) where the vertical location is located higher than the receiving module.

The above step to determine a position of pulmonary tumor according to another example of the present invention, can detect volume location linking the above dip to the transmission location with a virtual line and judge it as the location of tumor based on double dip obtained from the amplitude information measured from each offset locations and location information of the transmitter.

The present invention may have the following advantages.

According to an example of the present invention, it is possible to determine the location of tumor with relatively simple method without giving a risk to human body.

In order words, there is no risk of exposure to radiation and it is possible also not only to perform procedure and determine the location of tumor to patients with vulnerable respiratory organ or some diseases. In addition, no additional signal treatment process is needed, so the time required for determining the location is short and it is affected less by the measurement noise.

### BRIEF DESCRIPTION OF THE DRAWINGS

FIG. 1 is a state diagram showing use of the device to determine a position of pulmonary tumor using electromagnetic wave according to the present invention.

FIG. 2 is a diagram illustrating the electromagnetic wave receiving part according to the present invention.

FIG. 3 is a block diagram showing the electromagnetic wave transmitter and the device to determine a position of pulmonary tumor using electromagnetic wave according to another example of the present invention.

FIG. 4 is a flowchart illustrating the method to determine a position of pulmonary tumor according to another example of the present invention.

FIGS. 5A and 5B are views showing characteristics obtained from amplitude and phase information of electromagnetic wave received at the time of diagnosing lung caner;

FIG. 6 is a diagram illustrating a case of detecting pulmonary tumor according to one example of the present invention.

FIG. 7 is a diagram illustrating a case of determining a location of a pulmonary tumor according to one example of the present invention.

FIG. 8 is a diagram illustrating a case of detecting pulmonary tumor using vertical location offset according to another example of the present invention.

FIG. 9 is a diagram illustrating a case of determining a position of pulmonary tumor using vertical location offset according to another example of the present invention.

### DETAILED DESCRIPTION OF THE INVENTION

Various modifications and embodiments can be made in the present invention, and reference will be made in detail to the preferred embodiments of the present invention, examples of which are illustrated in the accompanying drawings. However, it should also be understood that embodiments are not limited by any of the details of the foregoing description, but rather should be construed broadly within its spirit and scope and it is intended that the present invention cover modifications and variations of this invention provided they come within the scope of the appended claims and their equivalents.

Though terms including ordinal numbers such as a first, a second, etc. may be used to explain various components, the components are not limited to the terms. The terms are used only for the purposed of distinguishing one component from another component.

For instance, a first component may be referred to as a second component, or similarly, the second component may be referred to as the first component, without departing from the scope of the present invention. A term ‘and/or’ is used to include a combination of a plurality of disclosed items or one of the items.

In a case it is mentioned that a certain component is “connected” or “accessed” to another component, it may be understood that the certain component is directly connected or accessed to the another component or that a component is interposed between the components. On the contrary, in case it is mentioned that a certain component is “directly connected” or “directly accessed” to another component, it should be understood that there is no component therebetween.

Terms used in the present invention is to merely explain specific embodiments, thus it is not meant to be limiting. A singular expression includes a plural expression except that two expressions are contextually different from each other. In the present invention, a term “include” or “have” is intended to indicate that characteristics, figures, steps, operations, components, elements disclosed on the specification or combinations thereof exist. Rather, the term “include” or “have” should be understood so as not to pre-exclude existence of one or more other characteristics, figures, steps, operations, components, elements or combinations thereof or additional possibility.

Except that they are not differently defined, all terms used in the present invention including technical or scientific terms have the same meanings with terms that are generally understood by those skilled in the art related to the field of the present invention. The terms same as those of which are defined in a general dictionary should be understood that the terms have meanings same as contextual meanings of the related art. And, as long as the terms are not definitely defined in the present invention, the terms are not interpreted as ideal or excessively formal meanings.

Description will now be given in detail of the exemplary embodiments, with reference to the accompanying drawings. For the sake of brief description with reference to the drawings, the same or equivalent components will be provided with the same reference numbers, and description thereof will not be repeated.

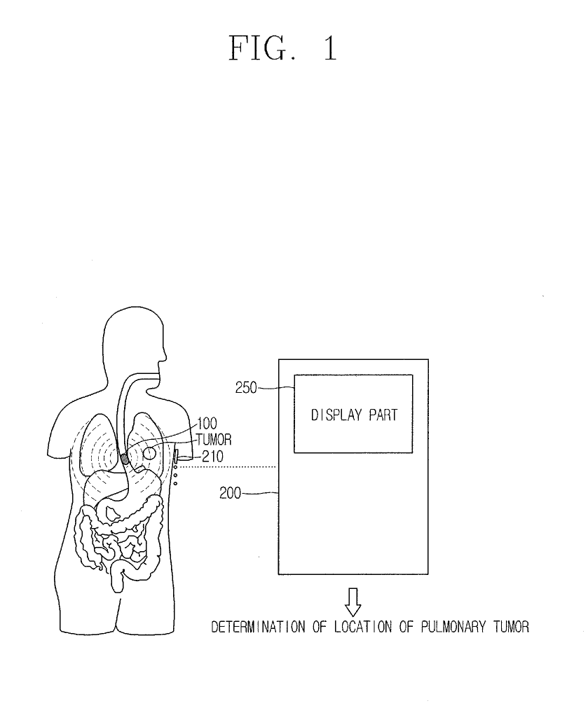

FIG. 1 is a state diagram showing use of the device to determine a position of pulmonary tumor using electromagnetic wave according to the present invention.

As shown in FIG. 1, the device (200) to determine a position of pulmonary tumor using electromagnetic wave according to the present invention includes a receiving part (210) to receive electromagnetic wave generated from a electromagnetic wave transmitter (100), and an display part (250) to display the information processed in the above device.

The above electromagnetic wave transmitter (100) may connect to external devices via wire or wireless. Especially, the above electromagnetic wave transmitter (100) should be ingested through the oral cavity, so it is made in a capsule shape to facilitate swallowing it.

The above electromagnetic wave transmitter (100) may move along with the human digestive tract vertically. The electromagnetic wave generated in the electromagnetic wave transmitter (100) is received by the receiving part (210). The above electromagnetic wave has a shape of continuous wave.

The electromagnetic wave information received at the above receiving part (210) is transmitted to the device for determining a position of pulmonary tumor using electromagnetic wave via wired or wireless path and the measuring part of the above device measures at least one of size or phase information of the electromagnetic wave. The controller of the above device (200) can determine a position of pulmonary tumor based on the location information of the above transmitter (100).

The above display part (250) can display at least one of size or phase information of the electromagnetic wave measured by the above measuring part and indicates a position of pulmonary tumor decided by the above controller. In addition, it is possible to displays the location information of the transmitter (100) detected by the controller.

The method to determine location of the above transmitter includes several methods comprising a method using signal intensity received by a receiving module, a method to determine location information obtained from the transmitter via communication, and a method to insert a magnetic to the transmitter and use a magnetic sensor in exterior.

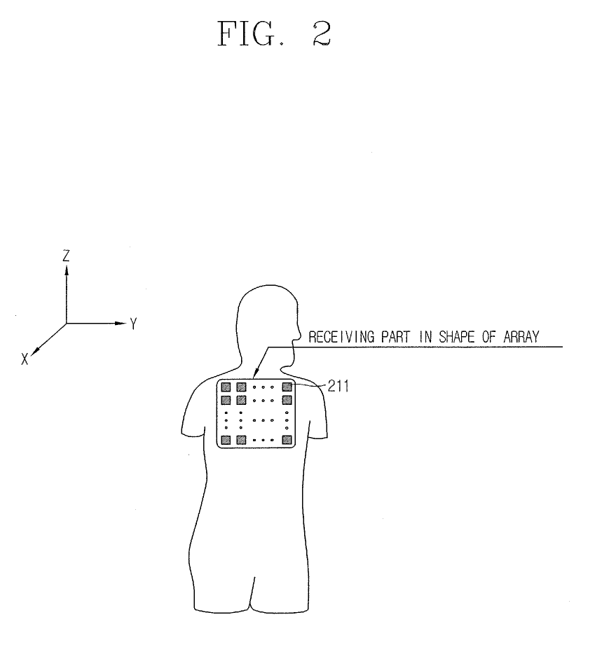

FIG. 2 is a diagram illustrating the electromagnetic wave receiving part according to the present invention.

In order to explanation of the present invention, x, y, and z axis are defined. The coordinate axis vertical to the surface is defined as z axis. And 2 coordinate axes parallel to the surface are defined as x and y axis. Especially, the coordinate axis in direction of crossing the front and the rear side of human body is defined as x axis and the coordinate axis vertical to the above x-axis is defined as y axis.

As shown in FIG. 2, the electromagnetic wave receiving part (210) according to the present invention includes at least one receiving module (211). The above receiving module (211) can receive the electromagnetic wave generated in the above electromagnetic wave transmitter (100).

In order to receive the electromagnetic wave generated in the above electromagnetic wave transmitter (100), the above receiving module (211) may be attached directly to the surface of upper half of the human body or located near to the human body. Therefore, the receiving part (210) may receive the electromagnetic wave, moving up and down and left and right occasionally.

The above receiving module (211) may be distributed in an array shape (z axis and y axis), maintaining a certain intervals. The intervals and number of the above receiving modules may be adjusted properly to the necessity of user. Especially, for the receiving module (211), n×n shaped array is general, but 1×n or N×1 shaped arrays may be possible occasionally and it is possible also to switch on only receiving modules (211) on the specific line or row and switch off the receiving modules (211) on the remaining lines or rows.

It is desirable for proper lung examination that the above electromagnetic wave receiving modules (211) are distributed broader than the range surrounding the human lung.

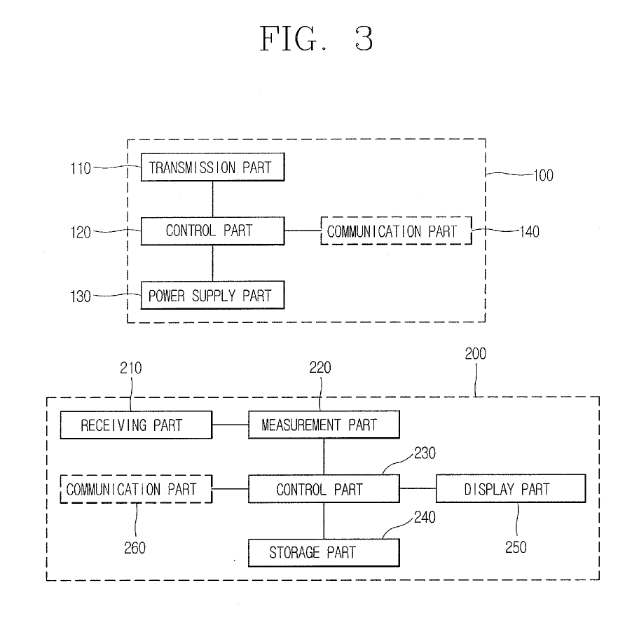

FIG. 3 is a block diagram showing the electromagnetic wave transmitter and the device to determine a position of pulmonary tumor using electromagnetic wave according to another example of the present invention.

The above electromagnetic wave transmitter (100) includes a transmitting part (110), a controller (120), and a power supply part (130). The above transmitting part (110) may include a broadband antenna and generate electronic magnetic wave, sweeping broadband frequency. The above sweeping of broadband frequency may change diagnosable size of cancer and in this present invention is aiming at diagnosing a cancer in early stage (i.e. below 1 cm of lung cancer). Especially, the above electromagnetic wave may have a shape of continuous wave.

The above controller (120) generally controls overall operation of the electromagnetic wave transmitter (100). For instance, it can control electromagnetic wave band generated by the transmitting part (110) or sweeping of broadband frequency and control output or interruption of electromagnetic wave according to prescribed period or conditions. In addition, it can control location and direction of the electromagnetic wave transmitter (100).

The power supply part (130) provides electric power necessary for operation of each component from internal power source under control of the controller (120). The above power supply part (130) may be constituted with battery.

The above electromagnetic wave transmitter (100) may include a communication part (140) also. The above communication part (140) can make the above electromagnetic wave transmitter (100) communicate with external devices via wired or wireless path. Through wired or wireless communication of the above transmitter (110), It is possible to receive signals necessary for control of the above transmitter (110) or transmit information related to the above transmitter to external devices.

The signals necessary for control of the transmitter (100) may include signals for controlling sweep in frequency range, output or period of electromagnetic wave, and wavelength of frequency, or signals for controlling location of the transmitter (100). The information related to the above transmitter (100) may include location information of the transmitter or status information to check up its malfunction.

The above device to determine a position of pulmonary tumor using electromagnetic wave (200) may include a receiving part (210), a measuring part (220), a controller (230), and a storage part (240). The above receiving part (210) receives the electromagnetic wave generated in the above transmitter (100). In addition, the above receiving part (210) may include a broadband antenna. Occasionally, it may include a magnetic sensor to obtain location information of the above transmitter (100).

The above measuring part (220) may obtain size information and phase information of the above received electromagnetic wave. By the above measuring part (220), it is possible to confirm size information and phase information of the above electromagnetic wave received by each receiving part.

The above controller (230) determines a position of pulmonary tumor and control overall operation of the receiving part (210) and the communication part (260) based on the results measured by the measuring part and the location information of the transmitter.

The methods to obtain the location information of the transmitter include: a method to detect it based on signal intensity of the electromagnetic wave received by the receiving part (210); a method to receive the location information acquired by the transmitter (100) through the communication part (260); and a method to use signals acquired through the magnetic sensor of the receiving part (210).

The storage part (240) may store a program for operation of the controller or data measured by the measuring part of electromagnetic wave. The storage part may include at least one selected from the group comprising: flash memory type, hard disk type, multimedia card micro type, card type memories (i.e. SD or XD memory), RAM (random access memory), SRAM (static random access memory), ROM (read-only memory, EEPROM (electrically erasable programmable read-only memory), PROM (programmable read-only memory), magnetic memory, magnetic disk and optical disk. The above device to determine a position of pulmonary tumor using electromagnetic wave may operate, associated with web storage to perform storing function of the above storage part (240) on internet.

The display part (250) may display or demonstrate information to be processed in the device to determine a position of pulmonary tumor using electromagnetic wave. For example, it may display at least one of size or phase information of electromagnetic wave measured by the above measuring part (220) or indicates the location of tumor judged by the controller (230). Or, it may display the location information of the transmitter (100) detected by the controller. The display part (250) may include at least one selected from the group comprising: LCD (liquid crystal display), TFT LCD (thin film transistor-liquid crystal display), and OLED (organic light-emitting diode), PDP (plasma display panel), and MDT (Multi Display Tube). Or it may include a printer capable of output on a paper.

The above device to determine a position of pulmonary tumor (200) may include a communication part (260) also. The above communication part may enable the above device to determine a position of pulmonary tumor (200) to communicate with the above electromagnetic wave transmitter (100) via wired or wireless route. Through this communication, the above device to determine a position of pulmonary tumor (200) may transmit signals necessary for controlling the above transmitter (100) or receive information related on the above transmitter (100).

The signals necessary for control of the transmitter (100) may include signals for controlling sweep in frequency range, output or period of electromagnetic wave, and wavelength of frequency, or signals for controlling location of the transmitter (100). The information related to the above transmitter (100) may include location information of the transmitter or status information to check up its malfunction.

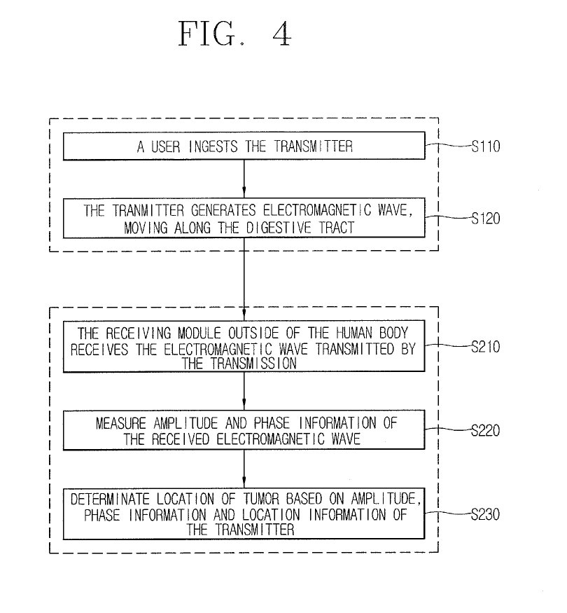

FIG. 4 is a flowchart illustrating the method to determine a position of pulmonary tumor according to another example of the present invention.

At first as shown in FIG. 4, a use ingests the electromagnetic wave transmitter (100) via oral cavity for tumor diagnosis (S110).

The above transmitter (100) can generate electromagnetic wave in shape of continuous wave, moving along the digestive tract (S120). Sweep in the frequency range of the above generated electromagnetic wave may be controlled.

The electromagnetic wave generated in the transmitter may be transmitted to human body. And, the electromagnetic wave receiving module (211) on outside of the human body may receive the above electromagnetic wave (S210).

After receiving the electromagnetic wave, it is possible to measure amplitude and phase information of the received electromagnetic wave (S220). The above received electromagnetic wave may be in state of which characteristics have been converted by characteristics of lung and cancer.

The above controller (230) can determine the location of tumor based on the measurement results and location information of the electromagnetic wave transmitter (100) (S230).

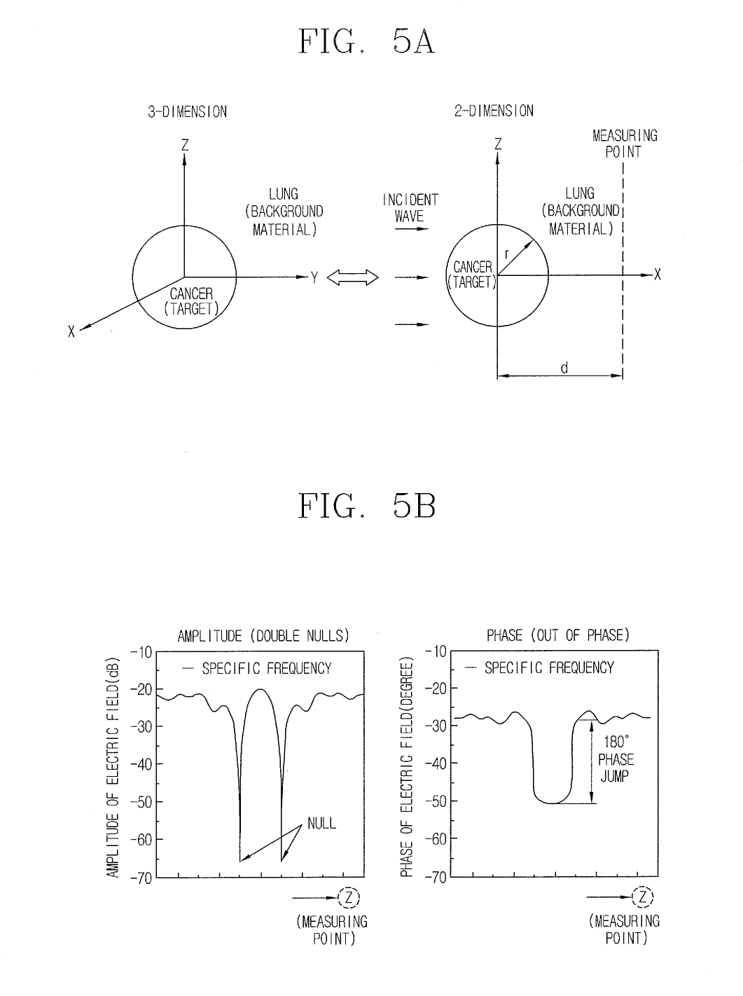

FIGS. 5A and 5B are views showing characteristics obtained from amplitude and phase information of electromagnetic wave received at the time of diagnosing lung caner;

FIG. 5A shows a case where cancer (target) exists on lung (background material). Referring to FIG. 5A, electromagnetic wave is incident onto a receiving part positioned on a measuring point, on each point on the Z-axis. The electromagnetic wave may proceed toward the X-axis, and may reach the receiving part positioned on the measuring point, via the lung (background material) and the cancer (target). In this case, the electromagnetic wave may have a size and a phase thereof changed, due to its diffusion property. That is, a characteristic of the received electromagnetic wave is determined according to whether the electromagnetic wave passes through the background material or the target.

FIG. 5B is a view showing a size and a phase of received electromagnetic wave when cancer exists. The graph was obtained by displaying a position in a vertical direction (Z-axis) of each receiving part on the X-axis, and by displaying a size and a phase of electromagnetic wave received by the receiving part on the Y-axis.

Distinctive phenomena were observed in size and phase of the received electromagnetic wave, according to a shape and a size of lung cancer (diffusion body), and a frequency of applied electromagnetic wave. That is, if lung cancer exists, double dip or double null can be detected from the above size graph. If lung cancer exists, phase jump can be detected from the above graph. The phase jump indicates a phenomenon that a large phase difference occurs on a specific part. From the above graph, it can be observed that a phase difference of about 180° occurs.

When the electromagnetic wave transmitter (100) moving along digestive organs (esophagus) in a vertical direction transmits electromagnetic wave and the receiving modules (211) receive the electromagnetic wave generated from the electromagnetic wave transmitter (100), the controller may check a characteristic of the received electromagnetic wave to thus diagnose lung cancer. That is, if double dip or double null is checked from the size graph of the received electromagnetic wave, and if phase jump is checked from the phase graph of the received electromagnetic wave, the controller may determine lung cancer.

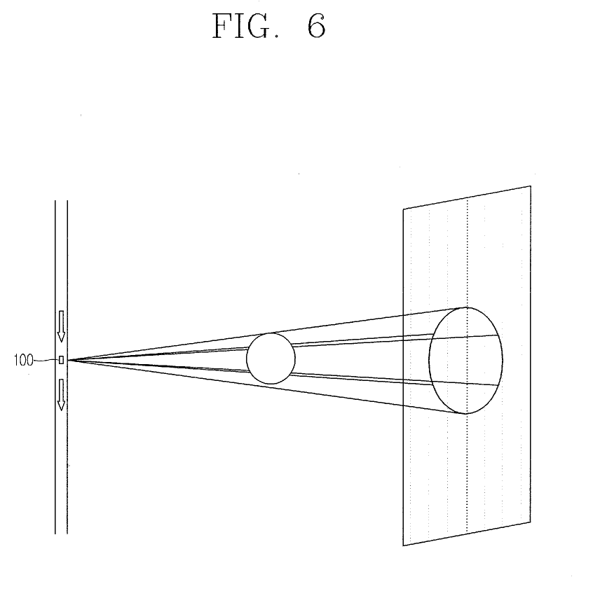

FIG. 6 is a diagram illustrating a case of detecting pulmonary tumor according to one example of the present invention.

The electromagnetic wave transmitter (100) discharges electromagnetic wave, moving along with the digestive tract. Occasionally, it may be possible also to fix the above transmitter (100) on a specific location and move the receiving part (210) vertically or laterally.

The electromagnetic wave discharged from the above transmitter (100) is received by the receiving part located in outside of the human body and having multiple receiving modules (211). As shown in FIG. 6, when there is a tumor (target) in the lung (background material), the electromagnetic wave reaches a receiving part located in the measuring point, passing through the tumor. Wherein, the above electromagnetic wave may change its size and phase by diffusion property of the electromagnetic signals. In other words, it is suggested that depending on whether the above electromagnetic wave passes through the background medium or the target, characteristics of the above received electromagnetic wave is determined. Besides, it is possible to observe unique phenomena in size and phase of the received electromagnetic wave, according to shape and size of the tumor, the diffusion body, and frequency relationship of the electromagnetic wave. Namely, when a tumor exist, it is possible to confirm dip or phase jump from the received electromagnetic wave signals. The above phase jump is a phenomenon that large phase difference occurs at a specific point.

It is possible to measure amplitude and phase information by the measuring part (220) from the received electromagnetic wave and determine existence of tumor from change of the measurement values.

As shown in FIG. 6, it is found that if the electromagnetic wave transmitted by the above transmitter passes through a tumor, a dip with a shape similar to the shape of tumor will appear on the receiving part. For example, when the shape of tumor is a spherical shape, a dip may appear in a circular arrange on the receiving part.

All of the dip signals may be used as information to determine the location of tumor or only a double dip on the specific row or column may be use as information for determining the location of tumor.

For the above method using only double dip, a receiving part (210) with 1×n or n×1 array of receiving modules may be used. In the receiving part (210) with n×n array, it is possible to obtain a double signal by switching on specific row or column and switching off remaining row or column and detect only 2 specific dip signals among the received several dip signals and use them in calculation for determining the location of tumor.

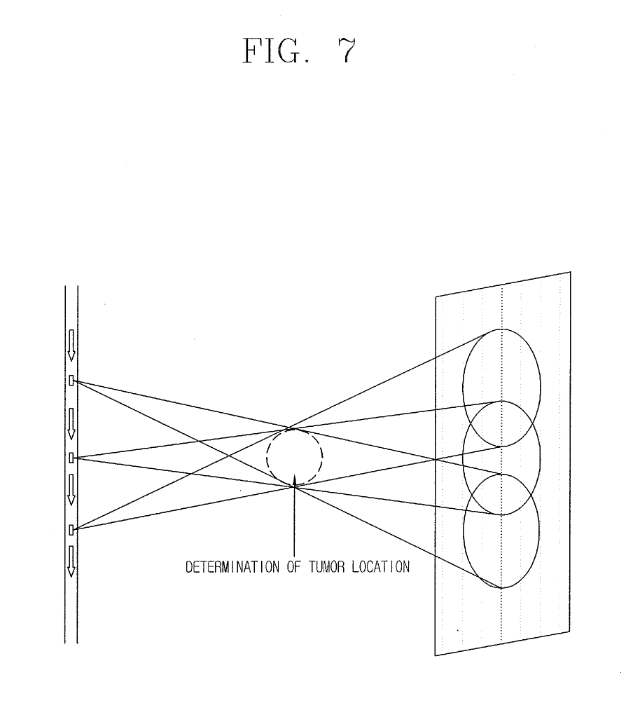

FIG. 7 is a diagram illustrating a case of determining a location of a pulmonary tumor according to one example of the present invention.

The electromagnetic wave transmitted by the above transmitter (100) is passed through tumor and received by the receiving part (210) and its size and phase information are measured by the measuring part (220).

In case that the transmitter (100) measures amplitude information from the electromagnetic wave transmitted by more than 2, preferably 3 locations and confirms a dip, the controller (230) performs calculation linking the above dip and the transmitted location with a virtual line. Through the above calculation for more than 2 locations, the region where the virtual lines are overlapped is detected and the detected region is considered as the location of tumor.

When the transmission locations of the transmitter (100) where the dip appears increases, it is possible to determine the location of tumor more accurately but the amount of calculation and the time required for determination of location may increase also. Therefore appropriate selection may be needed.

As explained in FIG. 6, it is possible to detect only double dip signals and use them in calculation for determining tumor location.

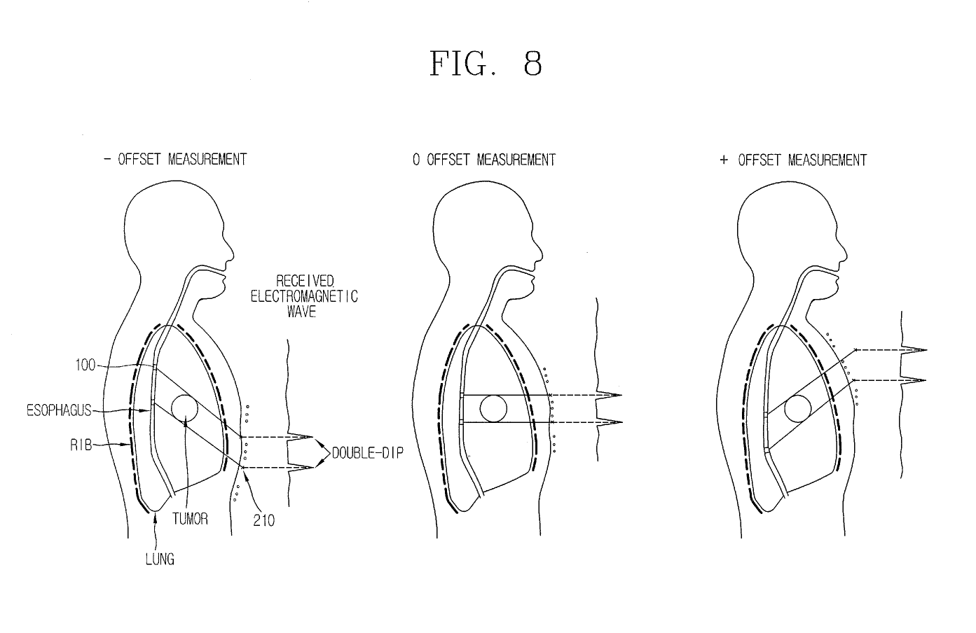

FIG. 8 is a diagram illustrating a case of detecting pulmonary tumor using a vertical location offset according to another example of the present invention.

Offset means difference from reference point. In the present invention, the case that the electromagnetic wave transmitter is higher than the tumor in vertical location is defined as negative offset (−offset) and the case that the transmitter (100) is lower than the tumor is defined as positive offset (+offset) on the basis of tumor location.

The electromagnetic wave transmitter (100) transmits electromagnetic wave, moving along with the digestive tract. Double deep detected by the electromagnetic wave transmitted by the transmitter (100) when it is located higher than the tumor and passing through top and bottom of the tumor, is referred as negative offset (−offset). Double deep detected by the electromagnetic wave transmitted by the transmitter (100) when it is located same to the tumor and passing through top and bottom of the tumor, is referred as zero offset (0 offset). And double deep detected by the electromagnetic wave transmitted by the transmitter (100) when it is located lower than the tumor and passing through top and bottom of the tumor, is referred as positive offset (+offset).

The method to detect each offset value, as explained in FIG. 6, may be done by using an array receiving module in a shape of 1×n or n×1, measuring in state of switching on only some receiving modules in specific row or column, and detecting only specific dips among several dips detected.

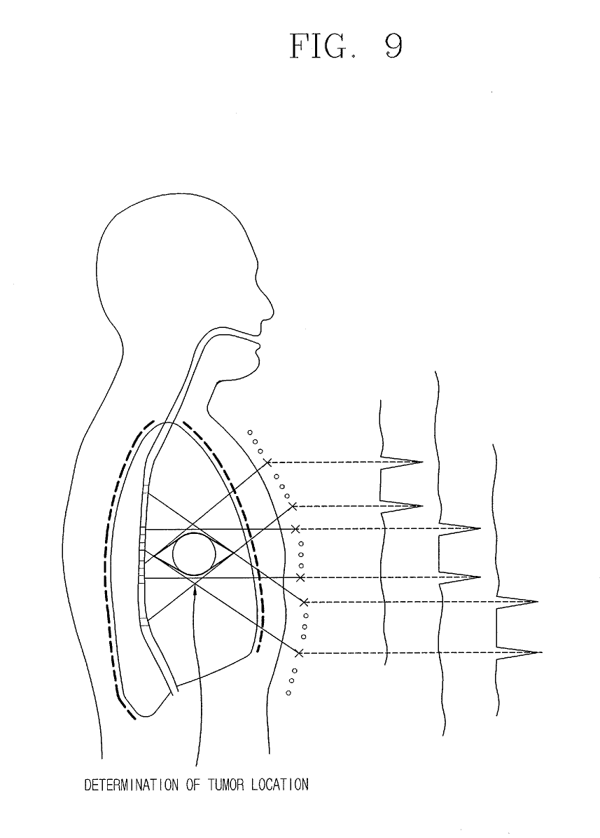

FIG. 9 is a diagram illustrating a case of determining a position of pulmonary tumor using vertical location offset according to another example of the present invention.

The calculation to link the double dip detected in negative offset, (−offset), zero offset (0 offset), and positive offset (=offset) and transmission location where the dip appears with virtual line is performed by the controller (230). Through the calculation, the region where the virtual line is overlapped is detected and the detected region is considered as the location of tumor.

The method for determining a position of lung cancer (or pulmonary tumor) will be explained in more detail.

Firstly, the transmitter may transmit electromagnetic wave to the receiving modules while moving to a lower end of a lung cancer-occurred part from an upper end, on the positive offset (+offset) position. The electromagnetic wave may include amplitude and phase information.

The controller may detect a double dip based on amplitude information corresponding to the electromagnetic wave (refer to FIGS. 5A and 5B). For instance, the shape of amplitude of a signal received based on electromagnetic wave may be a double dip. The controller may also detect a position of the double dip. For instance, the position of the double dip may indicate a position on the surface of the human body where the double dip has been detected (or a position on the surface of the human body from which electromagnetic wave including the double dip is emitted).

Alternatively, the position of the double dip may indicate two positions, because the double dip may have two dip points (or minimized points).

The controller may detect a phase jump based on phase information corresponding to the electromagnetic wave (refer to FIGS. 5A and 5B). The controller may also detect a position of the phase jump. For instance, the position of the phase jump may indicate a position on the surface of the human body where the phase jump has been detected, or a position on the surface of the human body from which electromagnetic wave having the phase jump is emitted.

In such manner, the controller may detect a double dip or a phase jump, based on electromagnetic waves transmitted while the transmitter moves to a lower end from an upper end of a lung cancer-occurred part at the zero offset (0 offset) or the negative offset (−offset), respectively.

According to an embodiment, the controller may generate virtual lines based on positions of a plurality of double dips, positions of a plurality of phase jumps, and positions of the transmitter. Here, the positions of the double dips, the positions of the phase jumps, and the positions of the transmitter are detected based on the +offset, zero offset and −offset positions.

For instance, in case of the positive offset (+offset), the controller may generate a virtual straight line which connects a position of the transmitter which is moving toward a lower end of pulmonary tumor from an upper end, with a position on the surface of the human body where electromagnetic wave which generates a double dip reaches. In such manner, the controller may generate a virtual straight line corresponding to the zero offset, and a virtual straight line corresponding to the negative offset (−offset). In this case, as shown in FIG. 9, the controller may determine a volume position as a position of lung cancer (or pulmonary tumor), the volume position enclosed by virtual straight lines generated from the respective offset positions.

The above described method to determine the location of tumor according to another example may be used individually or in combination. In addition, the steps composing an example of the present invention may be used with the steps composing another example individually or in combination.

In addition, the method described in the above can be realized within a record device able to read with computer or similar device using software, hardware, or their combinations.

For hardware type realization, the above mentioned method may be realized by using at least one of ASICs (application specific integrated circuits), DSPs (digital signal processors), DSPDs (digital signal processing devices), PLDs (programmable logic devices), and FPGAs (field programmable gate arrays, processors, controllers, micro-controllers, microprocessors, and other function performing.

By the software type realization, the procedures and functions described in the present invention may be realized by separate software modules. The above software modules may be materialized with software codes written by proper program language. The above software codes can be stored in the storage part and performed by the processor.

As the present features may be embodied in several forms without departing from the characteristics thereof, it should also be understood that the above-described embodiments are not limited by any of the details of the foregoing description, unless otherwise specified, but rather should be construed broadly within its scope as defined in the appended claims, and therefore all changes and modifications that fall within the metes and bounds of the claims, or equivalents of such metes and bounds are therefore intended to be embraced by the appended claims.

What is claimed is:

1. A device for diagnosing lung cancer using electromagnetic wave, comprising:
a receiving part to receive electromagnetic wave output from a transmitter ingested through the oral cavity and moving along with the digestive tract;
a measuring part to measure amplitude and phase information from the received electromagnetic wave; and
a controller to diagnose lung cancer based on the measured amplitude and phase information.

2. The device of claim 1, wherein the controller determines lung cancer in a case where at least one double dip or at least one double null is detected from the measured amplitude information.

3. The device of claim 1, wherein the controller determines lung cancer in a case where phase jump is detected from the measured phase information.

4. The device of claim 1, wherein the controller determines a position of pulmonary tumor based on the measured amplitude information, phase information and position information of the transmitter.

5. The device of claim 1, further comprising a communication part configured to transmit, to the transmitter, a signal for controlling an electromagnetic wave transmitting operation.

6. The device of claim 5, wherein the communication part receives a position and state information of the transmitter inside the human body.

7. The device of claim 1, wherein the receiving part includes a plurality of receiving modules which are contact or located near the surface of the human body and arranged in an array.

8. The device of claim 7, wherein when a dip is detected from the amplitude information measured from multiple transmission locations, the controller detects a volume location which connects the dip to the transmission location with a virtual line and determines it as the location of tumor.

9. The device of claim 7, wherein the controller acquires first amplitude information measured at a positive offset (+offset) where a vertical location of the transmitter is located lower than the receiving module, second amplitude information measured at a zero offset (0 offset) where a vertical location of the transmitter is located same to the receiving module, and third amplitude information measured at a negative offset (−offset) where a vertical location of the transmitter is located higher than the receiving module,
wherein the controller detects positions of double dips corresponding to the positive offset, the zero offset and the negative offset, respectively, based on the first amplitude information, the second amplitude information and the third amplitude information, and
wherein the controller determines a volume position as a position of lung cancer, the volume formed by virtual straight lines which connect the detected positions of the double dips to a position of the transmitter, respectively.

10. The device of claim 1, wherein the transmitter transmits electromagnetic wave in a prescribed period.

11. The device of claim 5, wherein the transmitter includes a communication part for wired or wireless communication for exchange of the above information.

12. A method for diagnosing lung cancer, comprising:
receiving electromagnetic wave generated from a transmitter ingested through the human oral cavity and moving along with the digestive tract;
measuring amplitude and phase information from the received electromagnetic wave; and
diagnosing lung cancer based on the measured amplitude and phase information.

13. The method of claim 12, wherein if at least one double dip or at least one double null is detected from the measured amplitude information, it is determined that lung cancer has occurred.

14. The method of claim 12, wherein if phase jump is detected from the measured phase information, it is determined that lung cancer has occurred.

15. The method of claim 12, further comprising determining a position of pulmonary tumor based on the measured amplitude information, the measured phase information, and position information of the transmitter.

16. The method of claim 12, further comprising transmitting, to the transmitter, a signal for controlling an electromagnetic wave transmission operation.

17. The method of claim 16, further comprising receiving a position and state information of the transmitter inside the human body, from the transmitter.

18. The method of claim 12, wherein in the step of determining a position of pulmonary tumor, when a dip is detected from the amplitude information measured from multiple transmission locations, a volume location which connects the dip to the transmission location with a virtual line is detected, and it is determined as the location of tumor.

19. The method of claim 12, wherein in the step of receiving electromagnetic wave output from the transmitter, electromagnetic wave is received from a positive off set (+offset) where a vertical location of the transmitter is located lower than a receiving module, a zero offset (0 offset) where the vertical location is located same to the receiving module, and a negative offset (−offset) where the vertical location is located higher than the receiving module.

20. The method of claim 19, wherein in the step of determining a position of pulmonary tumor, a volume location which connects the dip to the transmission location with a virtual line is detected, based on position information of a double dip and the transmitter, the information acquired from amplitude information measure from each offset position, and the volume location is determined as a position of pulmonary tumor.
